SEQUENCE LISTING (6 sequences)

SEQ ID NO: 1 - DNA, 1895 bp, Homo sapiens
atatgagctcaggcctggctgggctccaggagaatctttccacaaggcaagagataacacaaagtcaaggtgaagatcagggtagccctttaaaaggcctccttgtgccctagagttgccacagctcttctactccactgctgtctatcttgcctgccggcacccagccaccatgtgggagctcgtggctctcttgctgcttaccctagcttatttgttttggcccaagagaaggtgccctggtgccaagtaccccaagagcctcctgtccctgcccctggtgggcagcctgccattcctccccagacacggccatatgcataacaacttcttcaagctgcagaaaaaatatggccccatctattcggttcgtatgggcaccaagactacagtgattgtcggccaccaccagctggccaaggaggtgcttattaagaagggcaaggacttctctgggcggcctcaaatggcaactctagacatcgcgtccaacaaccgtaagggtatcgccttcgctgactctggcgcacactggcagctgcatcgaaggctggcgatggccacctttgccctgttcaaggatggcgatcagaagctggagaagatcatttgtcaggaaatcagtacattgtgtgatatgctggccacccacaacggacagtccatagacatctcctttcctgtcttcgtggcggtaaccaatgtcatctccttgatctgcttcaatacctcctacaagaatggggaccctgagttgaatgtcatacagaattacaatgaaggcatcatagacaacctgagcaaagacagcctggtggacctagtcccctggttgaagattttccccaacaaaaccctggaaaaattaaagagccatgttaaaatacgaaatgatctgctgaataaaatacttgaaaattacaaggagaaattccggagtgactctatcaccaacatgctggacacactgatgcaagccaagatgaactcagataatggcaatgctggcccagatcaagactcagagctgctttcagataaccacattctcaccaccataggggacatctttggggctggcgtggagaccaccacctctgtggttaaatggaccctggccttcctgctgcacaatcctcaggtgaagaagaagctctacgaggagattgaccagaatgtgggtttcagccgcacaccaactatcagtgaccgtaaccgtctcctcctgctggaggccaccatccgagaggtgcttcgcctcaggcccgtggcccctatgctcatcccccacaaggccaacgttgactccagcatcggtgagtttgctgtggacaagggcacagaagttatcatcaatctgtgggcgctgcatcacaatgagaaggagtggcaccagccggatcagttcatgcctgagcgtttcttgaatccagcggggacccagctcatctcaccgtcagtaagctatttgcccttcggagcaggacctcgctcctgtataggtgagatcctggcccgccaggagctcttcctcatcatggcctggctgctgcagaggttcgacctggaggtgccagatgatgggcagctgccctccctggaaggcatccccaaggtggtctttctgatcgactctttcaaagtgaagatcaaggtgcgccaggcctggagggaagcccaggctgagggtagcacctaaaggctgtaactcacagcccctgtccaccctatgtggccccacaacacagatttagagatacaaccccccacccttctccgccattcttccctactcccaacccactctgccttctttttcagcttgtggcaatgccagtgatgtgcataaacagtttttttttttccataaaaaaaaaaaaaaaaaaaaaaaaaaa

SEQ ID NO: 2 - protein, 508 aa, Homo sapiens
MWELVALLLLTLAYLFWPKRRCPGAKYPKSLLSLPLVGSLPFLPRHGHMHNNFFKLQKKYGPIYSVRMGTKTTVIVGHHQLAKEVLIKKGKDFSGRPQMATLDIASNNRKGIAFADSGAHWQLHRRLAMATFALFKDGDQKLEKIICQEISTLCDMLATHNGQSIDISFPVFVAVTNVISLICFNTSYKNGDPELNVIQNYNEGIIDNLSKDSLVDLVPWLKIFPNKTLEKLKSHVKIRNDLLNKILENYKEKFRSDSITNMLDTLMQAKMNSDNGNAGPDQDSELLSDNHILTTIGDIFGAGVETTTSVVKWTLAFLLHNPQVKKKLYEEIDQNVGFSRTPTISDRNRLLLLEATIREVLRLRPVAPMLIPHKANVDSSIGEFAVDKGTEVIINLWALHHNEKEWHQPDQFMPERFLNPAGTQLISPSVSYLPFGAGPRSCIGEILARQELFLIMAWLLQRFDLEVPDDGQLPSLEGIPKVVFLIDSFKVKIKVRQAWREAQAEGST

SEQ ID NO: 3 - DNA, 20 bp, Artificial Sequence
  Synthetic
ttcgtatgggcaccaagact

SEQ ID NO: 4 - DNA, 20 bp, Artificial Sequence
  Synthetic
gttgttggacgcgatgtcta

SEQ ID NO: 5 - DNA, 20 bp, Artificial Sequence
  Synthetic
gttcgacagtcagccgcatc

SEQ ID NO: 6 - DNA, 19 bp, Artificial Sequence
  Synthetic
ggaatttgccatgggtgga

(19) United States
(12) Patent Application Publication
(10) Pub. No.: US 2014/0100127 A1
(43) Pub. Date: Apr. 10, 2014

(54) LIVER CANCER DIAGNOSIS MARKER AND USE THEREOF

(71) Applicants: Mujun Zhao, Shanghai (CN); Feng Wang, Shanghai (CN); Zhen Xing, Shanghai (CN); Liang Da, Shanghai (CN); Ying Xu, Shanghai (CN); Zaiping Li, Shanghai (CN)

(72) Inventors: Mujun Zhao, Shanghai (CN); Feng Wang, Shanghai (CN); Zhen Xing, Shanghai (CN); Liang Da, Shanghai (CN); Ying Xu, Shanghai (CN); Zaiping Li, Shanghai (CN)

(73) Assignee: Shanghai Institutes for Biological Sciences, Chinese Academy of Sciences, Shanghai (CN)

(21) Appl. No.: 14/008,250

(22) PCT Filed: Mar. 30, 2012
(86) PCT No.: PCT/CN2012/073356

§ 371 Date: Dec. 11, 2013

(30) Foreign Application Priority Data
Mar. 31, 2011 (CN) 201110079327.9

(51) Int. Cl. C12Q 1/68; G01N 33/574

(52) U.S. Cl. CPC C12Q 1/6886; G01N 33/57438
USPC 506/9; 435/6.12; 536/24.5; 530/389.7; 536/24.33; 435/7.1

(57) ABSTRACT

A marker for the detection of liver cancer and application of the marker thereof. The application includes use of cytochrome p450 family 17 subfamily A polypeptide 1 (CYP17A1 protein) in the preparation of diagnostic reagents or kits for the detection of liver cancer kit. Studies have shown that CYP17A1 expression levels are higher in liver cancer tissues than in the adjacent healthy tissues, and the amount of CYP17A1 in sera of liver patients is significantly higher than that of healthy human population. Therefore, CYP17A1 can be used as a marker for the diagnosis of liver cancer (especially serological diagnosis).

### BACKGROUND OF INVENTION

1. Field of the Invention

The present invention relates to the field of oncology and diagnostics. More specifically, the present invention relates to a diagnostic marker for liver cancer and its use.

2. Background Art

Cytochrome p450 family 17 subfamily A polypeptide 1 (referred to as “CYP17A1”). This protein is also known as 17 alpha-hydroxylase/17,20 lyase, belongs to cytochrome P450 enzyme system (cytochrome P450c17a enzyme), and is composed of 508 amino acids.

CYP17A1 protein is mainly localized on endoplasmic reticulum and has the activity of steroid 17alpha monooxygenase, 17alpha-hydroxylase, and 17,20 lyase. It is a key enzyme in synthetic pathway of steroid hormones, participating in the synthesis of including progesterone, mineralocorticoid, sugar (adrenal) corticosteroids, androgens, estrogens, etc. CYP17A1 gene mutations are associated with non-dependent type steroid 17alpha-hydroxylase deficiency, l7alpha-hydroxylase and 17,20-lyase double deficiency, pseudohermaphroditism, adrenal hyperplasia, etc. CYP17A1 knock-out mice are embryonic lethal (Bair SR; Mellon SH, Deletion of the mouse P450c17 gene causes early embryonic lethality., Mol Cell Bid 2004). Its function in sex hormone synthesis pathway is mainly to convert pregnenolone and progesterone into 17-OH hydroxylated forms, thereby produce, respectively, DHEA and androstenedione, and finally, produce, respectively, androgenic and oestrogenic sex steroids (Chung et al., 1987; Kagimoto et al., 1988; Van Den Akker et al., 2002).

Studies related to CYP17A1 have mainly focused on the enzymatic activity in adrenal glands and gonads and the function in anabolism of cholesterol and steroid. No association of CYP17A1 with liver cancer has been reported.

Liver cancer is a common malignant cancer in our nation, ranked third in cancer incidence, ranked second in mortality rate. Hepatocellular carcinoma, HCC, is the most common type of liver cancer.

Liver cancer patients in China accounts for 54% of total cases in the world. It is easier for male than female to have the disease. Currently, 5-year survival rate for liver cancer patients is less than 5%. Approximately, 549,000 patients die of this disease every year. And, incidence of this disease has been in upward trend year after year (obtained from WHO Mortality Rate Database). Therefore, increasing effort on liver cancer prevention will have great significance in decreasing mortality rate.

Currently, diagnosis of liver cancer depends mainly on imaging examination, liver biopsy histological examination, and laboratory examination. Imaging diagnosis plays an important role in liver cancer diagnosis. But, it has some limitations in diagnosing small hepatocellular carcinoma and in distinguishing benign from malignant nodules. Based on cirrhosis, benign lesions of regenerative nodules in liver and poorly developed nodules, etc. are relatively common, and have some overlaps with imaging features of liver cancer. It is still very difficult to distinguish between small benign and malignant lesions in liver by radiological examination. Compared to liver pathology, CT diagnosis of liver cancer is less sensitive. Invasive histopathological examination is main method for diagnosing liver cancer. Even with the very good fine needle aspiration, it, however, has relatively higher false-negative rate due to limited materials collected. And, there is a danger of causing tumor spread and needle tract seeding. Therefore, in clinic, there still remains a need of highly sensitive liver cancer-specific serum markers to distinguish between benign and malignant lesions in liver, or to perform follow-up in high-risk group for improving early diagnosis rate of liver cancer.

Early diagnosis of liver cancer is the most important factor for improving patient survival rate. Currently, alpha fetoprotein, AFP, is main diagnostic liver cancer serum marker used in clinic. However, sensitivity is only 40% ˜65% and specificity 76% ˜96%. Although AFP plays a positive role in liver cancer diagnosis, but sensitivity and specificity are not satisfactory. Moreover, the ratio of AFP-negative in new cases is increasing.

Therefore, it is an urgent priority to search for new liver cancer serum markers having diagnostic or combined diagnostic value, and is a key for early detection and early treatment of HCC. Thus, providing gene or protein specifically and highly expressed in liver cancer tissue and serum will have important diagnostic and therapeutic implications. Developing serum specific markers, which can be used for the detection or determination of liver cancer, is urgently needed in the field.

### SUMMARY OF INVENTION

The object of the present invention is to provide a specific serum marker, which can be used for the detection or determination of liver cancer and the use thereof.

The first aspect of the present invention provides a use of a gene, an mRNA, a cDNA of cytochrome p450 family 17 subfamily A polypeptide 1 (CYP17A1 protein), which is used as a marker for the detection of liver cancer; or in the preparation of a reagent or a kit for the detection of liver cancer. More preferably, the detection is serum detection.

In another preferred embodiment, the reagent includes antibody, primer, probe, nucleic acid microarray (such as DNA microarray) or protein microarray.

The second aspect of the present invention provides a use of cytochrome p450 family 17 subfamily A polypeptide 1 (CYP17A1 protein) or its specific antibody, which is used in the preparation of a diagnostic reagent or a kit for the detection of liver cancer. More preferably, the detection is serum detection.

The third aspect of the present invention provides a diagnostic kit used for the detection of liver cancer, wherein the kit comprises:

(a) an anti-cytochrome p450 family 17 subfamily A polypeptide 1 (CYP17A1 protein) antibody; and/or

(b) a primer or a pair of primers specifically amplifying CYP17A1 mRNA or CYP17A1 cDNA.

In another preferred embodiment, the kit further comprises a label or an instruction, wherein the label or the instruction indicating that the kit is used for the detection or the diagnosis of liver cancer.

In another preferred embodiment, the anti-CYP17A1 protein antibody is a monoclonal antibody or a polyclonal antibody.

The fourth aspect of the present invention provides a method for the detection of liver cancer, wherein the method comprises:

a) preparing a test sample of a subject;

b) detecting an expression level of cytochrome p450 family 17 subfamily A polypeptide 1 gene (CYP17A1) in the test sample, and comparing the detected expression level with a reference value, wherein the expression level of CYP17A1 is higher than the reference value indicating that the subject has liver cancer, or has a higher risk of having liver cancer than a healthy human population

In another preferred embodiment, the test sample is a tissue sample, a blood sample, a serum sample, or a body fluid sample.

In another preferred embodiment, the reference value is an expression level of CYP17A1 in a non-liver cancer sample.

In another preferred embodiment, the detecting step b comprises detecting an amount of CYP17A1 mRNA, or an amount of CYP17A1 cDNA; and/or detecting an amount of CYP17A1 protein.

In another preferred embodiment, the step b comprises performing the detection by RT-PCR or PCR method.

In another preferred embodiment, the detecting step b comprises performing detecting using an anti-CYP17A1 protein antibody.

In another preferred embodiment, the detecting step b is achieved by an enzyme-linked immunoassay (ELISA method) or a time-resolved immunofluorescence method (TRFIA method).

In another preferred embodiment, the anti-CYP17A1 protein antibody is a monoclonal antibody or a polyclonal antibody (such as anti-serum).

In another preferred embodiment, wherein the method further comprises evaluating an expression level of another liver cancer marker in the test sample.

In another preferred embodiment, the other cancer marker includes: alpha-fetoprotein AFP, AFP isoform AFP-L3, serum fucosidase AFU, heparan sulfate proteoglycan 3 GPC3, abnormal prothrombin DCP , transglutaminase enzyme II (GGT II), or a combination thereof.

In another preferred embodiment, the method further includes evaluating the expression of alpha-fetoprotein (AFP) in the test sample.

The fifth aspect of the present invention provides a use of cytochrome p450 family 17 subfamily A polypeptide I (CYP17A1 protein) or its specific antibody, wherein it is used in the preparation of a diagnostic reagent or a kit for the serum detection of liver cancer.

In another preferred embodiment, the CYP17A1 protein or its specific antibody is coupling to or with a detectable marker.

In another preferred embodiment, the detectable marker is selected from the group: a chromophore, a chemiluminescent group, a fluorophore, an isotope, or an enzyme.

In another preferred embodiment, the diagnostic reagent is a monoclonal antibody.

In another preferred embodiment, the reagent is a protein microarray.

In another preferred embodiment, the nucleic acid microarray comprises a substrate and specific oligonucleotide probes for cancer-related genes spotted on the substrate, the specific oligonucleotide probes for cancer-related genes include probes that specifically hybridize with CYP17A1 polynucleotide (mRNA or DNA).

In another preferred embodiment, the protein microarray comprises a substrate and specific antibodies for cancer-related proteins spotted on the substrate, the specific antibodies for cancer-related proteins include specific anti-CYP17A1 antibodies.

In another preferred embodiment, the specific antibodies are monoclonal antibodies or polyclonal antibodies.

In another preferred embodiment, the serum detection is by ELISA, or double-antibody sandwich time-resolved fluorescence method (TRFIA method).

The sixth aspect of the present invention provides a diagnostic kit used for the detection of liver cancer, wherein the kit comprises a container, wherein the container comprising CYP17A1 protein or its specific antibody; and a label or an instruction, wherein the label or the instruction indicating that the kit is used for the serum detection or the serum diagnosis of liver cancer

In another preferred embodiment, the label or the instruction indicates the following: If serum concentration of CYP17A1≧70 ng/ml (preferably ≧80 ng/ml, more preferably >90 ng/ml, optimally ≧100 ng/ml) is detected in a subject, then the object has a greater risk of developing liver cancer than healthy human population.

In another preferred embodiment, the CYP17A1 protein or its specific antibody is coupling to or with a detectable marker.

In another preferred embodiment, the liver cancer includes hepatocellular carcinoma, especially primary hepatocellular carcinoma.

In another preferred embodiment, the CYP17A1 protein or its specific antibody is coupling to or with a detectable marker.

In another preferred embodiment, the detectable marker is selected from the group: a chromophore, a chemiluminescent group, a fluorophore, an isotope, or an enzyme.

In another preferred embodiment, the specific antibody is monoclonal antibody or polyclonal antibody.

The seventh aspect of the present invention provides a diagnostic kit used for the detection of liver cancer, wherein the kit comprises a container, wherein the container comprising a specific primer for a specific amplification of CYP17A1 mRNA or cDNA; and a label or an instruction, wherein the label or the instruction indicating that the kit is used for determining a risk of having liver cancer by quantitative detection of an expression level of CYP17A1.

In another preferred embodiment, the label or the instruction indicates the following: If a ratio of an amount of CYP17A1 mRNA detected in a subject to an amount of CYP17A1 mRNA detected in the general population is ≧1.5 (preferably ≧2.0, more preferably ≧2.5), then the object has a greater risk of developing liver cancer than healthy human population.

The eighth aspect of the present invention provides a use of cytochrome p450 family 17 subfamily A polypeptide 1 (CYP17A1 protein), wherein it is used as a marker for the serum detection of liver cancer.

The ninth aspect of the present invention provides a use of an antagonist of cytochrome p450 family 17 subfamily A polypeptide 1 (CYP17A1 protein), wherein it is used in the preparation of a medicament for the inhibition of liver cancer cell growth.

In another preferred embodiment, the antagonist comprises CYP17A1-targeting siRNA, antisense RNA, antibody, or a combination thereof.

The tenth aspect of the present invention provides an in vitro method of detecting whether CYP17A1 mRNA expression in liver tissue is abnormal, the method comprising the following steps:

A, determining a value of CYP17A1 mRNA in a test liver tissue by quantitative PCR using specific CYP17A1 primers;

B, comparing the value of CYP17A1 determined in step A with a value of CYP17A1 in healthy liver tissue, if the determined value is higher than the healthy value, then CYP17A1 expression in the tested liver tissue is abnormal.

The eleventh aspect of the present invention provides an in vitro method of detecting whether CYP17A1 protein expression in liver tissue is abnormal, the method comprising the following steps:

A, determining an amount of CYP17A1 protein in liver tissue by using specific anti-CYP 17A1 antibody;

B, comparing an amount of CYP17A1 determined in step A with an amount of CYP17A1 in the healthy liver tissue, if the determined protein amount is higher than the healthy amount, then CYP17A1 expression in the tested liver tissue is abnormal.

The twelfth aspect of the present invention provides an in vitro method of detecting whether CYP17A1 protein amount in serum is abnormal, the method comprising the following steps:

A, determining an amount of CYP17A1 protein in serum by using specific anti-CYP17A1 antibodies;

B, comparing an amount of CYP17A1 determined in step A with an amount of CYP17A1 in healthy human serum, if the determined protein amount is higher than the healthy amount, then CYP17A1 amount in the tested serum is abnormal.

It should be understood that, within the scope of the present invention, each technical feature of the present invention described above can be combined with each technical feature specifically described below (such as Examples), from which constitutes new or preferred technical solution. Due to space limitations, they are not individually described here.

### BRIEF DESCRIPTION OF DRAWINGS

FIG. 1. Results of differential expression of CYP17A1 mRNA in 33 pairs of liver cancer and adjacent tissue samples. In the figure, T represents liver cancer tissue and N represents adjacent tissue.

FIG. 2. Detection of CYP17A1 protein expression in liver cancer and adjacent tissue samples by Western blot method. In the figure, T represents liver cancer tissue and N represents adjacent tissue. β-actin is used as an internal reference.

FIG. 3. Immunohistochemical analysis of CYP17A1 protein expression in liver cancer patients and corresponding adjacent liver tissue samples. The figure shows representative pictures of one of the pairs of histochemical samples, 200× magnification, and a scale bar of 100 μm.

FIG. 4. Tissue microarray detection of differential CYP17A1 protein expression in liver cancer tissue and corresponding adjacent tissue samples of liver cancer patients. A, Immunohistochemical staining of CYP17A1 protein in tissue microarray. Left panel shows a pair of representative microarray spots of liver cancer and adjacent tissue. Right panel shows partially enlarged views of this pair of microarray spots, 200× magnification, and a scale bar of 100 μm. B, Comparison of differential CYP17A1 protein expression in 87 pairs of liver cancer and adjacent tissue samples. In the figure, T represents liver cancer tissue and N represents adjacent tissue.

FIG. 5. Analysis of diagnostic value of CYP17A1 protein amount in healthy human serum and in liver cancer patient serum as serological markers for liver cancer. A. Determination of CYP17A1 protein in healthy human serum and liver cancer patient serum by ELISA. Healthy represents healthy human serum and HCC represents liver cancer patient serum. B. ROC curve analysis on the value of CYP17A1 as a serological diagnostic marker for liver cancer. The larger the area encompassed by the curve, determines the better the value.

FIG. 6 shows CYP17A1 expression in HCC at different degree of differentiation. In the figure, T represents liver cancer tissue sample and N represents corresponding adjacent healthy tissue.

FIG. 7A shows the principle of measuring blood levels of CYP17A1 by ELISA.

Goat anti-human CYP17A1 polyclonal antibody serves as capture antibody and rabbit anti-human CYP17A1 polyclonal antibody as detection antibody.

FIG. 7B shows a standard curve used for ELISA measurement. The standard curve was generated by measuring OD values of a gradient with different concentrations of fold-diluted CYP17A1 protein standards (0 pg/ml, 156.25 pg/ml, 312.5 pg/ml, 625 pg/ml, 1250 pg/ml, 2500 pg/ml, 5000 pg/ml, 7500 pg/ml, 10000 pg/ml).

FIG. 8 shows CYP17A1 expression in 212 cases of serum samples from different groups detected by ELISA method.

FIG. 9 shows amount and analysis of CYP17A1 protein in serum samples of

AFP-negative and AFP-positive liver cancer patents. Samples include 45 cases of AFP-negative (AFP−) specimens, 70 cases of AFP-positive (AFP+) specimens, and 30 cases of healthy specimens.

FIG. 10 shows ROC curve comparative analysis on sensitivity and specificity of using CYP17A1 and AFP in liver cancer diagnosis.

FIG. 11 shows the expression of CYP17A1 and AFP in liver cancer serum samples, indicating that the ratio of high CYP17A1 expression in sera of liver cancer patients is greater than that of AFP. In the figure, 34.5 ng/ml concentration of CYP17A1 and 20 ng/ml concentration of AFP were used as cut-off points.

### DETAILED DESCRIPTION

Through extensive and in-depth studies, the present inventors, for the first time, unexpectedly found that CYP17A1 was highly expressed in liver cancer tissue and was expressed at low levels in healthy liver tissue, thus, can be used as markers for liver cancer. In addition, liver cancer cells also produce secretive CYP17A1 into blood.

Therefore, CYP17A1 serum concentration was positively correlated with a risk of having liver cancer in test subject. Thus, serum CYP17A1 can be used as a marker for the detection of liver cancer. On this basis, the present invention was completed.

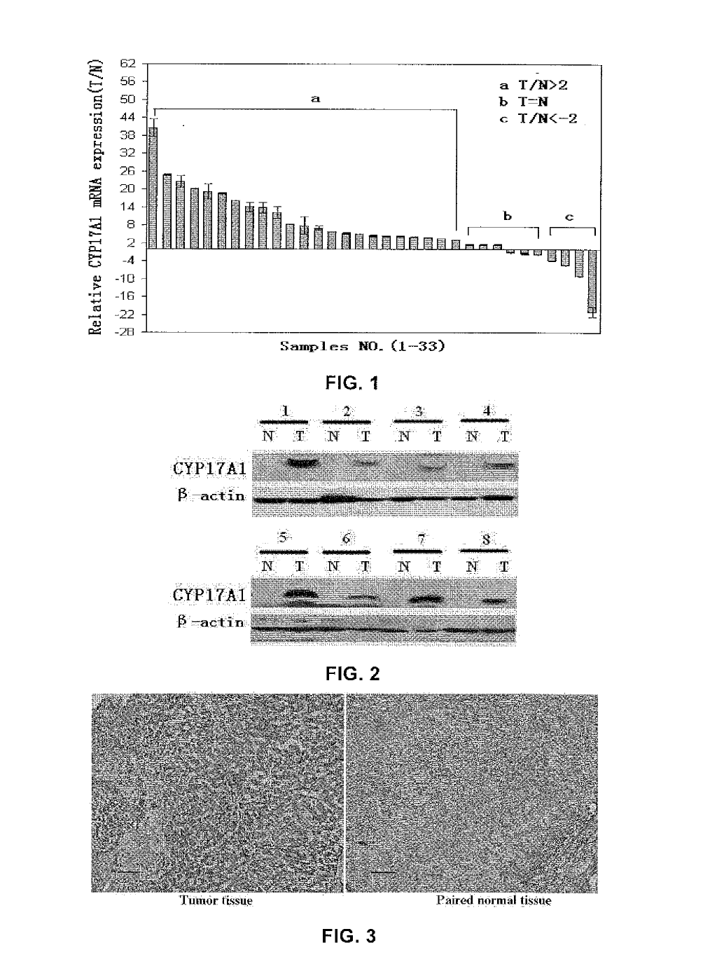

Specifically, the present inventors discovered that CYP17A1 gene was highly expressed in liver cancer by high-throughput gene expression microarray screening techniques. Then, the expression levels of CYP17A1 mRNA in 33 pairs of clinical liver cancer and adjacent tissue samples were detected by quantitative RT-PCR assay_{—} Results shows that 23 pairs had 2-fold and more higher expression levels of CYP17A1 mRNA in liver cancer than in adjacent tissue. The ratio of high expression is about 70% (23/33) [FIG. 1, Example 1].

The present inventors further detected the expression levels of CYP17A1 _{protein in} 60 pairs of liver cancer and adjacent tissue samples using Western blotting. Results proved that CYP17A1 protein was up-regulated in 44 pairs of samples. The ratio is about 73% (44/60) [FIG. 2, Example 3].

The present inventors also detected 5 pairs of liver cancer and adjacent tissue samples using immunohistochemical assay. Results showed that, in 5 pairs of histochemical samples, all expression in liver cancer tissue were significantly higher than that in corresponding adjacent tissues [FIG. 3, Example 4].

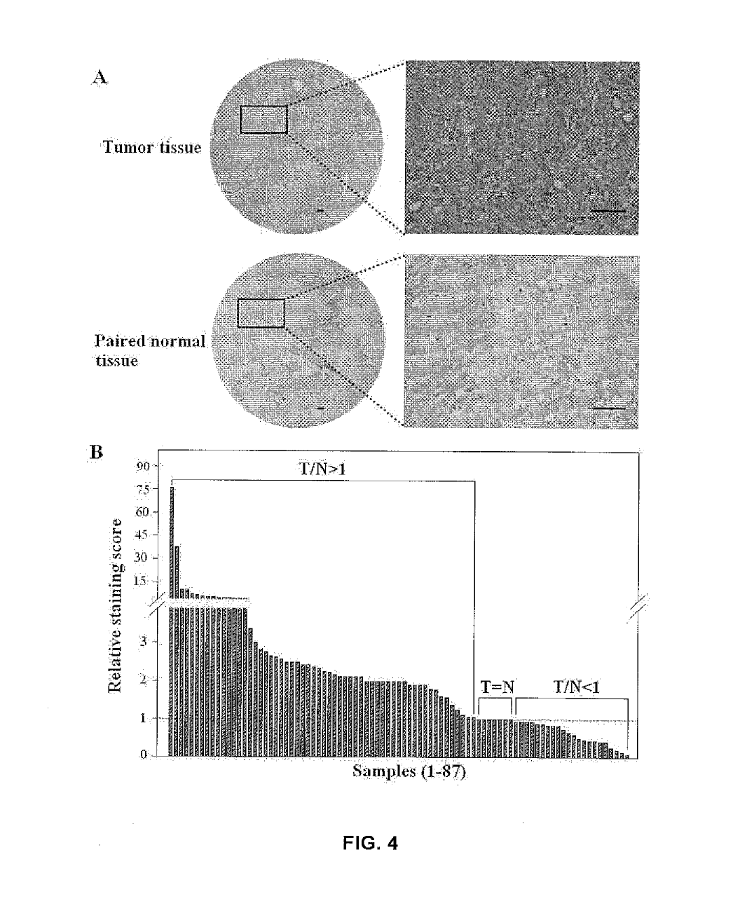

The present invention also performed immunohistochemistry detection on tissue microarray containing 87 pairs of human liver cancer and adjacent tissue samples. Results proved that 58 pairs had CYP17A1 protein expression in liver cancer tissue higher than that in adjacent tissue. The ratio is about 66.7% (58/87) [FIG. 4, Example 5].

These results indicate that, in clinical liver cancer tissue samples, CYP17A1 mRNA and CYP17A1 protein are highly expressed in liver cancer.

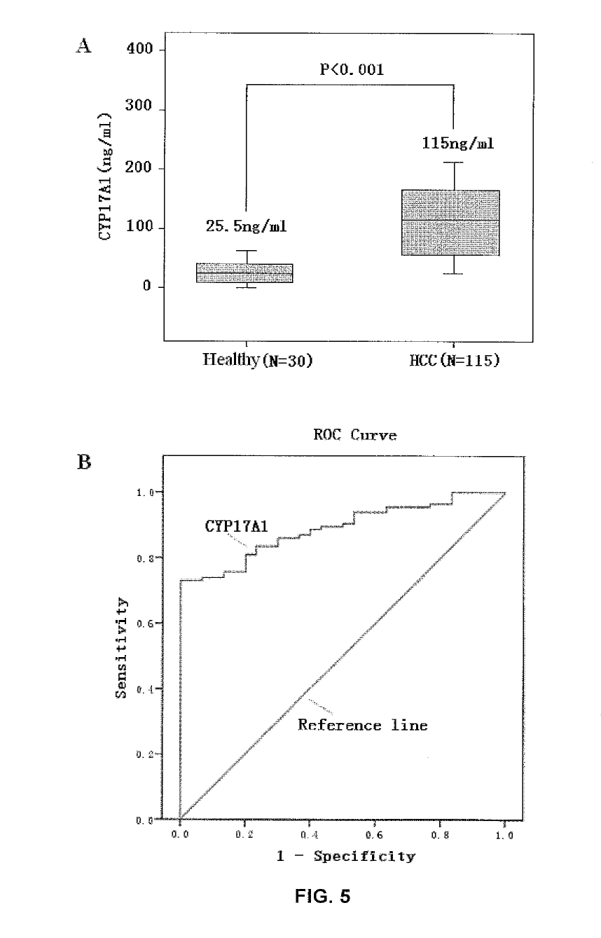

Further, the present inventors also detected the expression of CYP17A1 in human serum using ELISA. Results showed that CYP17A1 expression in sera of liver patients was significantly higher than that in healthy persons [FIG. 5, Example 6].

Average amount of CYP17A1 protein in sera of healthy persons (n=30 cases) was 25.5 ng/ml, and average amount in sera of liver cancer patients (n=115 cases) was 115 ng/ml. Statistical analysis indicates that high expression of CYP17A1 protein in sera of liver cancer patients is significantly different (P <0.001).

Sensitivity and specificity of detection could reach 86.1% and 70%, respectively, based on 95% confidence in the amount of CYP17A1 in healthy human serum, when 34.5 ng/ml concentration of CYP17A1 was cut-off point. ROC curve analysis, as shown in FIG. 5B, the larger the area under the ROC curve in the figure indicates the better the diagnostic value. The ROC curve area of CYP17A1 was 0.889, which was significantly larger than the reference curve area of 0.5 (P <0.001), indicating that CYP17A1 had good diagnostic value as serological molecular marker for liver cancer.

Sample

As used herein, the term “sample” or “specimen” refers to materials specifically related to a subject, from which specific information related to the subject can be determined, calculated, or inferred. Samples can be wholly or partly composed of biological materials originated from the subject. Samples can also be materials that have been in contact with the subject in some way. This way of contact causing the samples being tested to provide information related to the subject. Samples can also be materials that have been in contact with other materials. These other materials are not of the subject, but can cause the first material being tested afterward to determine the information related to the subject, for example, sample can be cleaning fluid of a probe or a surgical scalpel. Sample can be biological material sources in contact with those outside the subject, provided that one skilled in the art can still determine the information related to the subject.

Expression

As used herein, the term “expression” includes mRNA production from a gene or part of a gene, and includes production of RNA or protein encoded by a gene or part of a gene, and also includes appearance of test material related to the expression. For example, cDNA, binding ligands (such as antibodies) binding with genes or other oligonucleotides, proteins or protein fragments and chromogenic portions of binding ligands are all included in the scope of the term “expression.” Therefore, a slightest increase in density on immunoblot, such as Western blot, is also within the scope of the term “expression” based on molecular biology.

Reference Value

As used herein, the term “reference value” refers to, as compared with analysis results, statistically relevant value in specific results. In a preferred embodiment, the reference value is determined based on statistical analysis performed by comparing the expression of CYP17A1 protein with studies of known clinical results. Example section of the disclosure shows these types of studies. However, literature search and user experience in methods of the present disclosure can also be used to produce or adjust reference value. Reference value can also be determined by considering conditions and results specifically related to patient's medical history, genetics, age, and other factors.

Non-liver Cancer Samples

As used herein, the term “non-liver cancer samples” includes, but not limited to people who do not have liver cancer, and non-liver cancer tissue in liver cancer patients.

CYP17A1 Protein and Gene

In the present invention, the term “protein of the present invention,” “CYP17A1 protein,” “CYP17A1 polypeptide,” or “cytochrome p450 family 17 subfamily A polypeptide 1” are used interchangeably. All refer to a protein or a polypeptide having an amino acid sequence of cytochrome p450 family 17 subfamily A polypeptide 1 (NCBI protein SEQ ID NO: NP_{—}000093 or SEQ ID NO.: 2). They include CYP17A1 protein with or without starting methionine. In addition, the term also includes full length CYP17A1 and fragments thereof. CYP17A1 protein referred to in the present invention includes complete amino acid sequence thereof, secreted protein thereof, mutants thereof, and fragments thereof having functional activity.

In the present invention, the term “CYP17A1 gene,” “CYP17A1 polynucleotide,” or “cytochrome p450 family 17 subfamily A polypeptide 1 gene” are used interchangeably. All refer to nucleic acid sequences having human CYP17A1 nucleotide sequences. Full length CYP17A1 gene is 7003bp (NCBI GenBank Accession Number NC 000010.10), its transcript, full length mRNA sequence, is 1895bp (NCBI GenBank Accession Number NM 000102 or as shown in SEQ ID NO.: 1). It should be understood, when encoding identical amino acids, nucleotide substitutions in codons are acceptable. It should also be understood, when nucleotide substitutions produce conservative amino acid substitutions, the nucleotide conversions are also acceptable.

In the case that amino acid fragment of CYP17A1 was obtained, based on which the encoding nucleic acid sequence can be constructed, and specific probes can be designed according to the nucleotide sequence. Full length nucleotide sequence or fragments thereof can be usually obtained by PCR amplification method, recombination method, or synthetic method. For PCR amplification method, relevant sequences can be obtained by amplification according to nucleotide sequences of CYP17A1 disclosed by the present invention. Especially, primers are designed based on open reading frame sequences. And, commercially available cDNA libraries or cDNA libraries prepared based on conventional methods known by one skill in the art are used as template. When a sequence is relatively long, two or more PCR amplification are usually required, and fragments produced by each amplification are then spliced together in a correct order.

Once relevant sequences are obtained, large quantities of the relevant sequences can be obtained by recombinant method. This is typically performed by cloning them into vector, which are then transfected into cells. After cell proliferation, the relevant sequences are separated and obtained from host cells according to conventional method.

In addition, synthetic methods can also be used to synthesize relevant sequences, especially, when the length of fragments are relatively short. Typically, by first synthesizing multiple small fragments, and then spliced together to obtain fragments with very long sequences.

At present, DNA sequences encoding proteins of the present invention (or fragments thereof, derivatives thereof) can be obtained completely by chemical synthesis. DNA sequences can then be introduced into various currently available DNA molecules (such as vectors) and cells known in the art.

Through conventional recombinant DNA technology, recombinant CYP17A1 polypeptides can be expressed and produced using polynucleotide sequences of the present invention. In general, it includes the following steps:

(1). Using polynucleotide (or variant) encoding human CYP17A1 of the present invention, or using recombinant expression vector containing the polynucleotide to transform or transfect suitable host cells;

(2). Culturing host cells in suitable culture media;

(3). Isolating and purifying protein from culture media or cells.

In the present invention, polynucleotide sequence of CYP17A1 can be inserted into recombinant expression vector. In short, as long as they can replicate and are stable inside the host, any plasmids and vectors can be used. One important feature of expression vector, typically, is to have replication origin, promoter, marker gene, and translation control elements.

Methods known by one skilled in the art can be used to construct expression vector containing DNA sequence encoding CYP17A1 and suitable transcriptional/translational control signals. These methods include in vitro recombinant DNA techniques, DNA synthesis techniques, in vivo recombinant techniques, etc. The DNA sequence can be effectively linked to suitable promoter on expression vector to direct mRNA synthesis. Expression vector also includes ribosome binding sites used for translation initiation and transcription terminator.

In addition, expression vector preferably contains one or more selection marker genes to provide phenotypes used for selection of transformed host cells, such as dihydrofolate reductase used in eukaryotic cell culture for neomycin resistance and green fluorescent protein (GFP), or tetracycline or ampicillin resistance used in E. coll.

Vector containing the above-mentioned suitable DNA sequence and suitable promoter or control sequences can be used to transform suitable host cells, allowing them for protein expression.

Host cells can be prokaryotic cells, such as bacterial cells; or lower eukaryotic cells, such as yeast cells; or higher eukaryotic cells, such as mammalian cells. Representative examples include: E. coli, Streptomyces bacterial cells; fungal cells, such as yeast; plant cells; insect cells; animal cells, etc.

Conventional techniques well-known to one skilled in the art can be used to transform host cells using recombinant DNA. When the host is prokaryotic cell, such as E. coli, competent cells capable of absorbing DNA can be collected after exponential growth phase with CaCl₂ treatment. The procedures used are well known in the art. Another method is using MgCl₂. If necessary, transformation can be performed by using electroporation method. When the host is eukaryotic cell, the following DNA transfection method can be selected: calcium phosphate precipitation method, conventional mechanical methods, such as microinjection, electroporation, liposome encapsulation, etc.

The obtained transformant can be cultured using conventional method to express the polypeptide encoded by the gene of the present invention. Depending on host cell used, media used for culturing can be selected from various conventional culture media. Culturing is performed under conditions suitable for host cell growth. After host cells grown to appropriate cell density, suitable methods (such as temperature change or chemical induction) are used to induce promoter selected, and further culturing cells for a period of time.

In the above methods, recombinant polypeptide can be expressed inside the cell or on cell membrane, or be secreted outside the cell. If necessary, recombinant proteins can be separated and purified using its physical, chemical, and other characteristics by various separation methods. These methods are well known to one skilled in the art.

Examples of these methods include, but not limited to: conventional renaturation treatment, treatment with protein precipitation agent (salting method), centrifugation, osmotic breaking bacteria, ultra-treatment, ultracentrifugation, molecular sieve chromatography (gel filtration), adsorption chromatography, ion exchange chromatography, high performance liquid chromatography (HPLC), and various other liquid phase chromatography techniques, and a combination thereof.

Specific Antibodies

In the present invention, the term “antibody of the present invention” and “specific anti-CYP17A1 antibody” are used interchangeably.

The present invention also includes polyclonal antibodies and monoclonal antibodies with specificity for human CYP17A1 polypeptides, especially monoclonal antibodies. Here, “specificity” refers to antibodies capable of binding to human CYP17A1 gene products or fragments. Preferably, referring to those antibodies capable of binding to human CYP17A1 gene products or fragments, but not recognizing and binding to other un-related antigenic molecules. Antibodies of the present invention include those capable of binding to and inhibiting human CYP17A1 protein molecules, also include those antibodies that do not affect the function of human CYP17A1 protein. The present invention also includes those antibodies capable of binding to modified or unmodified form of human CYP17A1 gene products.

The present invention not only includes complete monoclonal or polyclonal antibodies, but also includes antibody fragments with immune activity, such as Fab′ or (Fab)₂ fragments; antibody heavy chain; antibody light chain; genetically engineered single chain Fv molecule (Ladner et al, U.S. Pat. No. 4,946,778); or chimeric antibodies, such as antibodies with binding specificity of mouse antibody, but still retaining portions derived from human antibodies.

Antibodies of the present invention can be prepared by a variety of techniques known to one skilled in the art. For example, purified human CYP17A1 gene products or fragments thereof with antigenicity can be administered to animals to induce polyclonal antibody production. Similarly, cells expressing human CYP17A1 protein or fragments thereof having antigenicity can be used to immunize animals for antibody production. Antibodies of the present invention can also be a monoclonal antibodies. This type of monoclonal antibodies can be prepared by using hybridoma technology (see Kohler et al, Nature 256; 495, 1975; Kohler, et al, Eur. J. Immunol. 6:511, 1976; Kohler, et al, Eur. J. Immunol. 6:292, 1976; Hammerling et al, In Monoclonal Antibodies and T Cell Hybridomas, Elsevier, N.Y., 1981). Antibodies of the present invention include antibodies capable of blocking the function of human CYP17A1 protein and antibodies not affecting the function of human CYP17A1 protein. Each type of antibodies of the present invention can be obtained using fragments or functional domains of human CYP17A1 gene products by conventional immunization techniques. These fragments or functional domains may be synthesized using recombination methods or polypeptide synthesizer. Antibodies binding to unmodified form of human CYP I 7A1 gene products can be produced by immunizing animals with gene products made by prokaryotic cells (e.g., E. coli); and antibodies binding to post-translationally modified form (such as glycosylated or phosphorylated proteins or polypeptides) can be produced by immunizing animals with gene products made by eukaryotic cells (such as yeast or insect cells).

Anti-human CYP17A1 protein antibodies can be used in immunohistochemical techniques to detect human CYP17A1 protein in specimens (especially serum samples).

Detection

Taking advantage of the features that CYP17A1 is present in serum and its close relationship with liver cancer, the present invention also provides a method of detection or determination of liver cancer, especially serological detection method.

In a preferred embodiment of the present invention, the present invention provides ELISA method and time-resolved immunofluorescence method (TRFIA) for the serum detection of CYP17A1.

Detection Kit

Based on a correlation between CYP17A1 and liver cancer, namely, CYP17A1 is highly expressed in liver cancer tissue and of high amount in sera of liver cancer patients. Therefore, CYP17A1 can be used as a serum diagnostic marker for liver cancer.

The present invention also provides a kit for the detection of liver cancer. It contains anti-CYP17A1 immunoglobulin or immunoconjugates, or the active fragments thereof; or contains primers for specific amplification of mRNA or cDNA of CYP17A1 of the present invention.

In another preferred embodiment, the present invention also provides diagnostic kit of CYP17A1, including: CYP17A1 mRNA diagnostic kit [Example 2] or CYP17A1 ELISA detection kit [Example 7].

100 cases have been completed experimentally by using human liver cancer serological diagnostic kit of the present invention. The positive rate was about 70%.

Subjects tested positive using human liver cancer serological diagnostic kit of the present invention had significantly higher risk of having liver cancer than healthy human population or general liver cancer patients.

Pharmaceutical Compositions

The invention also provides a pharmaceutical composition, which contains the above-mentioned CYP17A1 antagonist, and pharmaceutically acceptable carrier. The pharmaceutical composition can be used for inhibiting liver cancer cell growth_{—}

In the present invention, the antagonist includes CYP17A1-targeting siRNA, antisense RNA, antibodies, or a combination thereof. Further, the antagonist also includes small molecule compound capable of decreasing the expression or activity of CYP17A1.

Typically, CYP17A1 antagonist may be prepared in non-toxic, inert and pharmaceutically acceptable aqueous carrier medium, in which pH is usually about 5-8, preferably pH is about 6-8, although the pH values may vary according to the nature of materials prepared and illness treated. The prepared pharmaceutical composition can be administered through conventional means, including (but not limited to): intraperitoneal, intravenous, or topical administration.

Pharmaceutical composition of the present invention can be directly used for inhibiting liver cancer cell growth. In addition, it can also be used in combination with other cancer therapeutic agents.

Pharmaceutical composition of the present invention contain safe and effective amount of the above-mentioned CYP17A1 antagonist of the present invention and pharmaceutically acceptable carrier or excipient. This kind of carrier includes (but not limited to): saline, buffer, glucose, water, glycerol, ethanol, and a combination thereof Pharmaceutical preparations should match mode of administration. Pharmaceutical composition of the present invention may be prepared in injection form, for example, preparation using physiological saline or aqueous solution containing glucose and other auxiliary agents by conventional method. Pharmaceutical composition, such as injection, solution, should be prepared under sterile conditions. Dosage of active ingredients is an effective amount of treatment, for example, about 1 μg/kg body weight - about 5 mg/kg body weight per day. In addition, polypeptides of the present invention can be used in combination with other therapeutic agents.

When using pharmaceutical composition, administering a safe and effective amount of CYP17A1 antagonists of the present invention to mammal, in which the safe and effective amount is usually at least about 10 μg/kg body weight, and, in most cases, not exceeding about 8 mg/kg body weight, preferably the dosage is about 10 μg/kg body weight—about 1 mg/kg body weight. Of course, for specific dosage, factors involving administration route, patient health status, etc. should be considered. These are all within technical scope of skilled physicians.

Main advantages of the present invention include:

(A) Liver cancer is one of malignant tumors with the highest mortality rate. Early detection and early treatment are the most effective way to improve patient survival rate. Currently, due to few serum markers available for early diagnosis of liver cancer, patients found with cancer are mostly at advanced stages. The present inventors, for the first time, discovered CYP17A1 was a liver cancer serum marker, which can be used for early diagnosis of liver cancer.

(2) Provide a new method for the detection and determination of liver cancer by serum markers, which are helpful in early detection or auxiliary detection of liver cancer, thus, contributing to early diagnosis to take relevant therapeutic measures.

(3) Serum detection method is more convenient and rapid, and easier for patients to accept.

(4) The present invention also provides detection method and kit using CYP17A1 for the diagnosis of liver cancer, providing reliable assurance for specific application of CYP17A1.

With the following specific embodiments, the present invention is further illustrated. It should be understood that these embodiments are merely used to illustrate the present invention and are not used to limit the scope of the present invention. Experimental methods with un-specified conditions are usually in accordance with conventional conditions, such as conditions described in Sambrook et al, Molecular Cloning: A Laboratory Manual (New York: Cold Spring Harbor Laboratory Press, 1989), or in accordance with conditions recommended by manufacturers. Unless otherwise indicated, percentages and parts are percentages by weight and parts by weight.

Example 1

### Fluorescence Quantitative RT-PCR Detection of CYP17A1 mRNA Expression in Human Liver Cancer Tissue Samples

Test materials and preparation thereof: 33 cases of fresh samples of liver cancer and adjacent tissue thereof from liver patients were selected and stored in liquid nitrogen. Total RNA of each tissue sample was prepared by using TRIzol kit (Invitrogen Co.) according to methods recommended by the instructions. cDNA templates were prepared by reverse transcription.

Primers for CYP17A1 quantitative PCR and primers for GAPDH as internal reference were designed and synthesized:

Upstream primer sequence of CYP17A1 is:
[SEQ ID NO.: 3]
5′-TTCGTATGGGCACCAAGACT-3′;
Downstream primer sequence of CYP17A1 is:
[SEQ ID NO.: 4]
5′-GTTGTTGGACGCGATGTCTA-3′;
Upstream primer sequence of GAPDH is:
[SEQ ID NO.: 5]
5′-GTTCGACAGTCAGCCGCATC-3′;
Downstream primer sequence of GAPDH is:
[SEQ ID NO.: 6]
5′-GGAATTTGCCATGGGTGGA-3′;

Methods of operation: In 20 μl reaction system, to which were successively added 1 μl cDNA template (from test tissue samples), 10 μl SYBR Master Mix (purchased from Applied Biosystems Co.), 1 μl of each upstream and downstream primers (10 μM), finally deionized water to fill up to 20 μl. Then, PCR reaction was performed under the following conditions: PCR conditions for the determination of CYP17A1 was denaturation at 95° C. for 10 minutes, followed by 40 cycles, each cycle included 95° C. for 20 seconds, 60° C. for 20 seconds, 72° C. for 25 seconds. Same reaction conditions were used to determine GAPDH. PCR instrument used was 7500 fast quantitative PCR instrument of Applied Biosystems Co. Quantitative analysis software provided by the same company was used to perform analysis on the results.

Results:

As shown in FIG. 1, in 33 pairs of liver cancer and adjacent tissue samples tested, 23 pairs had 2-fold and more higher CYP17A1 mRNA expression in liver cancer than in adjacent tissue. The ratio is about 70%. Therefore, high expression of CYP17A1 mRNA in liver cancer was significant (p <0.001).

### Example 2

Preparation of CYP17A1 mRNA Detection Kit

As described in Example 1, high expression of CYP17A1 mRNA was closely associated with liver cancer diseases. Based on that, CYP17A1 mRNA detection kit can be prepared.

The kit contains:
Reagent 1, upstream primer of CYP17A1 with a concentration of 100 μM.
Reagent 2, downstream primer of CYP17A1 with a concentration of 100 μM.
Reagent 3, 2XPCR reaction solution, including Tag DNA polymerase, dNTP, magnesium ion, SYBR fluorescent dye. The reagent can be purchased from Applied Biosystems Co.
Reagent 4, nuclease-free water.
Reagent 5, a pair of primers for internal reference GAPDH, each with a concentration of 100 μM.

Operating Instructions: (steps)

(1) Prepare test samples by extracting mRNA from test samples and reverse transcribed into cDNA. Conventional methods or kits (e.g., TRIzol RNA extraction kit) can be used.

(2) Prepare PCR reaction solution according to the following system: cDNA template, 0.5-2 μl Reagent 1, 1 μl (final concentration 0.5 μM/μl) Reagent 2, 1 μl (final concentration 0.5 μM/μl) Reagent 3, 10 μl Reagent 4, for filling up to 20 μl Note: At the same time, prepare internal reference GAPDH PCR reaction solution according to the same system under the same conditions.

(3) Perform PCR reaction with fluorescence quantitative PCR instrument. PCR reaction conditions can be adjusted based on need. Recommended conditions are denaturation at 95° C. for 10 minutes, followed by 40 cycles, each cycle includes 95° C. for 20 seconds, 60° C. for 20 seconds, 72° C. for 25 seconds.

(4) Analyze experimental results and compare with healthy control tissue samples. 2-fold or more than 2-fold higher CYP17A1 mRNA expression levels than the healthy control are considered abnormal.

### Example 3

Western Blot Detection of CYP17A1 Protein Expression in Human Liver Cancer Tissue Samples

Test materials and preparation thereof: 60 cases of fresh samples of liver cancer and adjacent tissue thereof from liver cancer patients were selected and placed in liquid nitrogen and quickly ground into tissue fragments. Tissue fragments were dissolved in RIPA lysis buffer (50 mM Tris •HCl pH 7.4, 150 mM NaCl, 1% NP-40, 1% sodium deoxycholate, 0.1% SDS; 1 ml RIPA/0.1 g tissue sample), placed on ice for 30 min, centrifuged under the conditions of 15000 rpm/min, 4° C. for 20 min Supernatant was collected. Total protein quantification was performed using BCA protein quantification test kit (purchased from Shanghai Sangon Co., Ltd.) and aliquoted into 50 μg each, stored at −80° C. for use.

Methods of operation: 12% SDS-PAGE was performed on 50 μg of proteins from each sample. When bromophenol blue reached to the bottom of gel, proteins were transferred to nitrocellulose membranes (purchased from Amersham Biosciences Co.) using Bio-Rad membrane transfer equipment, and sealed in 5% skim milk at room temperature for 1 hour, followed by incubation with primary antibody of rabbit anti-human CYP17A1 polyclonal antibody (purchased from Proteintech Co., at 1:1000 dilution) at 4° C. overnight. After incubation, membranes were washed three times with TBST (50 mM Tris, 150 mM NaCl, 0.05% Tween 20, pH adjusted to 7.6 using HCl), each time for 10 min. Goat anti-rabbit secondary antibody conjugated with horseradish peroxidase (HRP) (purchased from Santa Cruz Biotechnology, 1:2000 dilution) was incubated at room temperature for 1 hour. Membranes were washed three times with TBST, each time for 10 minutes. Finally, protein bands were detected by developing X-ray film exposed to ECL chemiluminescence reagents (purchased from Pierce Co.). At the same time, β-actin was used as equal amount loading control (β-actin monoclonal antibody was purchased from Sigma Co., 1:2000 dilution).

Results:

In 60 pairs of liver cancer and adjacent tissue protein samples tested, CYP17A1 was up-regulated in 44 pairs of samples, the ratio was about 73%. Therefore, the presence of high expression of CYP17A1 protein in liver cancer was significant (P <0.001). This result was basically the same as the ratio of its mRNA up-regulation in liver cancer tissue (70%). FIG. 2 shows high expression of CYP17A1 in 8 pairs of liver cancer tissue samples.

### Example 4

Immunohistochemical Analysis of CYP17A1 Protein Expression in Human Liver Cancer Tissue

Test materials and preparation thereof: 5 cases of liver cancer and corresponding adjacent tissue samples from liver patients were selected and fixed with 4% paraformaldehyde under the conditions of 4° C. for one hour or overnight. Washed with PBS buffer three times, each time for 10 min to 1 hour. After that, samples were placed in 30%, 50% ethanol for 30 min each, and finally stored in 70% ethanol at 4° C. to finish fixing. When preparing tissue sections, fixed samples were first dehydrated through ethanol gradient, transparent in xylene, and then paraffin embedded under the conditions of 52-54° C. Sections were sliced by microtome, section thickness 4-10 μm, affixed to clean glass slides treated with poly-lysine, baked at 34° C. overnight, and then sealed and stored at 4° C.

Methods of operation: The prepared tissue sections were chosen, first de-waxed by xylene, rehydrated by ethanol gradient, and then 0.3% hydrogen peroxide was added at 37° C. for 20 minutes, removing endogenous peroxidase. Sections were immersed in pH 6.0 citric acid buffer solution. Antigen retrieval was performed by microwaving for 15 minutes, cooled; washed with PBS for 5 minutes x 3 times. Rabbit anti-human CYP17A1 polyclonal antibody (purchased from Proteintech Co., 1:200 dilution) was added and reacted at 37° C. for 1 hour, followed by incubation at 4° C. overnight. Washed with PBS for 5 minutes x 3 times. Ready-to-use goat anti-rabbit secondary antibody conjugated with HRP (purchased from Dako Co.) was added and reacted at 37° C. for 1 hour. Washed with PBS for 5 minutes×3 times. Developed using DAB substrate solution (purchased from Dako Co.), re-stained with hematoxylin, dehydrated in ethanol, transparent in xylene, and mounted with neutral balsam.

Results:

As shown in FIG. 3, CYP17A1 protein was mainly localized in cytoplasm, exhibiting diffuse distribution. In 5 pairs of histochemical samples tested, all expression was significantly higher in liver cancer tissue than in corresponding adjacent tissue. FIG. 3 shows representative pictures of one pair of histochemical samples, in which the darker the brown color, indicating the stronger the CYP17A1 protein expression.

Example 5, Using tissue microarray to detect CYP17A1 protein expression in human liver cancer tissue samples

Test materials: To further confirm high expression of CYP17A1 in clinical tissue of human liver cancer and to expand the scale of detection, the present embodiment used tissue microarray containing 200 spots of liver cancer tissue (purchased from Shanghai Biochip Co.) for immunohistochemical analysis. This microarray contained liver cancer and corresponding adjacent tissue from 87 cases of liver cancer patients, cancer and corresponding adjacent tissue from 13 cases of non-cancer patients (including 3 pairs of bile duct carcinoma and corresponding adjacent tissue, 6 pairs of adenocarcinoma and corresponding adjacent tissue, 3 pairs of hemangiomas and corresponding adjacent tissue, and 1 pair of squamous cell carcinoma and corresponding adjacent tissue).

Methods of operation: Immunohistochemistry was performed using standardized procedures, completed by the microarray company. Rabbit anti-human CYP17A1 polyclonal antibody (purchased from Proteintech Co.) was used as primary antibody, (HRP)-conjugated goat anti-rabbit antibody as secondary antibody (purchased from Santa Cruz Co.). Results of CYP17A1 protein expression in tissue microarray were independently analyzed by two pathologists. Tissue microarray analysis was based on mainly two indicators, percentage of stained cells (0-100%), and staining intensity (0-3 level system was used: 0, no staining; 1, weak staining; 2, moderate staining; 3, strong positive staining). Comprehensive assessment of these two indicators yielded immunostaining score results (immunostaining score equals to the stained cell percentage multiplied by the staining intensity).

Results:

As shown in FIG. 4A, left panel is representative pictures of CYP17A1 expression in one pair of liver cancer and adjacent tissue thereof in microarray spots. Right panel is partially enlarged views of the microarray spots (200× magnification). The figure shows CYP17A1 protein is highly expressed in liver cancer tissue, mainly localized in cytoplasm, exhibiting diffuse distribution. FIG. 4B shows difference in CYP17A1 protein expression in each pair of liver cancer (T) and corresponding adjacent tissue (N). T/N >1 is high expression in liver cancer; T/N <1 low expression in liver cancer, T/N=1 no difference in expression.

In 87 pairs of liver cancer tissue samples of tissue microarray, 58 pairs had higher CYP17A1 protein expression in liver cancer tissue than in adjacent tissue. The ratio was about 66.7%. Statistical analysis showed that, high CYP17A1 protein expression in liver cancer was significant (P <0.001).

### Example 6

ELISA Determination of CYP17A1 Protein Amount in Sera of Liver Cancer Patients and in Sera of Normal Healthy Persons

Test materials and preparation thereof: Blood samples from 30 cases of normal healthy persons and 115 cases of liver cancer patients were collected. All these samples came from Eastern Hepatobiliary Surgery Hospital. Let blood samples stand at room temperature for 2 hours, causing natural coagulation. Centrifuged under the condition of 2500 rpm/min, 4° C. for 20 min. Supernatant was carefully removed. If precipitate appeared during collection, it should be re-centrifuged. The obtained supernatant was serum samples, after aliquoted, placed at −80° C. for storage.

Methods of operation: Goat anti-human CYP17A1 polyclonal antibody (purchased from Santa Cruz Biotechnology, capture antibody) was diluted at 1:400 dilution in 1× ELISA coating buffer (purchased from KPL Co.) and 100 μl/well added to microtiter plate (purchased from Shanghai Mai Co.) at room temperature for 1 hour. Well liquid aspirated, dried; 300 μl of 1× BSA blocking solution (purchased from KPL Co.) was added to each well, and blocked at room temperature for 10 minutes. Well liquid aspirated, dried; serum test samples was diluted at 1:100 in 1× BSA blocking solution, 100 μl/well was added to microtiter plate, and incubated at room temperature for 1 hour or at 4° C. overnight. Well liquid aspirated, dried; plate was washed 5 times with 1 x ELISA washing solution (purchased from KPL Co.), about 400 μl/well, and soaked for 1-2 minutes each time. Rabbit anti-human CYP17A1 polyclonal antibody (purchased from Proteintech Co., detection antibody) was diluted at 1:2000 in 1× BSA blocking solution. 100 μl/well was added to microtiter plate and incubated at room temperature for 1 hour. Well liquid aspirated, dried; and the above plate washing steps were repeated. Goat anti-rabbit secondary antibody conjugated with horseradish peroxidase (purchased from Proteintech Co.) was diluted at 1:3000 in 1× BSA blocking solution. 100 μl/well was added to microtiter plate at room temperature for 1 hour. Well liquid aspirated, dried; and the above plate washing steps were repeated. 100 μl of ABTS substrate solution (purchased from KPL Co.) was added to each well, developed in dark for 10 minutes. 100 μl of stop solution (purchased from KPL Co.) was added to each well to stop reaction. Optical density of each well (OD value) was measured immediately at 405 nm wavelength using microtiter plate reader. Meanwhile, CYP17A1 recombinant protein standards (purchased from Proteintech Co.) were diluted according to the following concentration gradient: 0 pg/ml, 156.25 pg/ml, 312.5 pg/ml, 625 pg/ml, 1250 pg/ml, 2500 pg/ml, 5000 pg/ml, 10000 pg/ml, and OD values were measured, together with test serum samples under the same conditions, to produce standard curve.

Results:

As shown in FIG. 5A, average amount of CYP17A1 protein in sera of healthy persons (n=30) was 25.5 ng/ml. Average amount in sera of liver cancer patients (n =115) was 115 ng/ml. Statistical analysis indicated that the amount of CYP17A1 protein in sera of liver cancer patients was significantly higher than in sera of healthy persons (P <0.001).

ROC curve analysis was performed on CYP17A1 expression levels in sera of healthy persons and in sera of liver cancer patients. Results shown in FIG. 5B, in the figure, the larger the area under the ROC curve indicates the higher the diagnostic value. ROC curve area of CYP17A1 was 0.889, which was significantly greater than the reference curve area 0.5 (P <0.001), indicating that CYP17A1 has good diagnostic value as serological molecular marker for liver cancer. Based on 95% confidence interval of CYP17A1 amount in sera of healthy persons, and CYP17A1 concentration of 34.5 ng/ml was used as cut-off point, sensitivity and specificity of detection could reach 86.1% and 70%, respectively.

### Example 7

Preparation of CYP17A1 ELISA Test Kit

As described in Example 6, CYP17A1 protein can be secreted into sera of liver cancer patients, and that the amount in sera of liver cancer patients was significantly higher than in sera of healthy persons. Statistical analysis showed that, when 34.5 ng/ml concentration was used as determination cut-off point, detection error was at the minimum, and sensitivity and specificity at the best. Based on that, ELISA test kit can be prepared.

The kit contains:
A 96-well microtiter plate,
Reagent A, goat anti-human CYP17A1 polyclonal, dilute 1:400 before use.
Reagent B, rabbit anti-human CYP17A1 polyclonal antibody, dilute 1:2000 before use.
Reagent C, goat anti-rabbit secondary antibody conjugated with horseradish peroxidase, dilute 1:3000 before use.
Reagent D, human CYP17A1 recombinant protein standards, concentration is 1 mg/ml (0.1 ml volume).

Other optional reagents include ELISA coating buffer, ELISA blocking solution, ELISA washing solution, ELISA developing solution, ELISA stop solution.

Operating instructions: (steps)

(1) Prepare test serum samples, let whole blood samples stand at room temperature for 2 hours. Centrifuge at 2500 rpm/min for 20 min. Collect supernatant and place temporarily at 4° C. for testing or at −80 ° C. for storage.

(2) Take a microtiter plate, set standard wells, test sample wells and control wells. Add 100 μl of reagent A to each well. Incubate at room temperature for 1 hour.

(3) Aspirate well liquid, dry; add 300 μl of ELISA blocking solution to each well, block at room temperature for 10 minutes.

(4) Add 100 μl/well of test serum samples at 1:100 dilution in ELISA blocking solution to microtiter plate. Incubate at room temperature for 1 hour or at 4° C. overnight. Meanwhile, dilute reagent D according to the following concentration gradient: 0 pg/ml, 156.25 pg/ml, 312.5 pg/ml, 625 pg/ml, 1250 pg/ml, 2500 pg/ml, 5000 pg/ml, 10000 pg/ml. Measure OD values, together with test serum samples according to the same procedure, to produce standard curve.

(5) Aspirate well liquid, dry, wash plate 5 times with ELISA washing solution. Immerse for 1-2 minutes each time, about 400 μl/well, dry (can also lightly tap to pat dry liquid inside the well).

(6) Add 100 μl of reagent B to each well, incubate at room temperature for 1 hour.

(7) Aspirate liquid, dry, wash plate 5 times with ELISA washing solution, the same method as in step 5.

(8) Add 100 μl of reagent C to each well, incubate at room temperature for 1 hour.

(9) Aspirate liquid, dry, wash plate 5 times with ELISA washing solution, the same method as in step 5.

(10) Add 100 μl of ELISA developing solution to each well, react in dark at room temperature for 10 minutes.

(11) Add 100 μl of ELISA stop solution to each well, stop reaction; immediately measure optical density of each well (OD value) at 405 nm wavelength using microtiter plate reader.

(12) Graph standard curve, calculate sample concentration. Perform analysis using 34.5 ng/ml as cut-off point. Greater than or equal to the cut-off point is determined to be cancer serum samples. Smaller than the cut-off point is determined to be healthy samples.

### Example 8

Liver Cancer Detection Kit

A kit was prepared for use in the serological detection of liver cancer, the kit contained:

(a) a first container, and the following antibodies specifically targeting CYP17A1 located inside the container: goat anti-human CYP17A1 polyclonal antibody (can be purchased from Santa Cruz Co., capture antibody);

(b) and label or instruction, the label or instruction indicated that the kit was used for the detection or diagnosis of liver cancer; and

(c) optionally a second container, and detection antibody located inside the container: rabbit anti-human CYP17A1 polyclonal antibody (can be purchased from Proteintech Co., detection antibody)

The above-mentioned test kit was used for quantitative detection of CYP17A1 amount in unknown serum samples (145 cases, in which 115 cases were HCC patient samples) by ELISA method.

Results showed that, when 70 ng/ml was chosen as positive threshold value, 81 cases of liver cancer samples were assessed as CYP17A1-positive, with a positive rate of about 70%.

### Example 9

CYP17A1 Expression Levels Were Correlated with Degree of Liver Cancer Differentiation

In this embodiment, correlation between CYP17A1 protein expression in liver cancer and clinical pathological parameters was further analyzed.

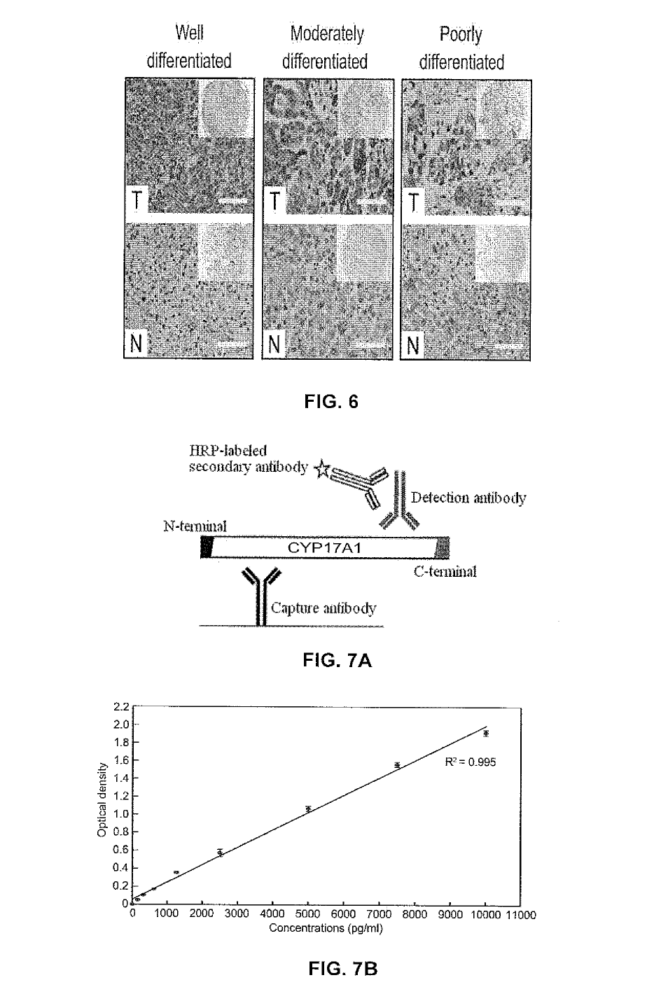

As results shown in Table 1, correlation was not found between CYP17A1 expression and age, gender, but the expression was significantly correlated with histological grades (P=0.036). In that, 01 was relatively better differentiated hepatocellular carcinoma (HCC), G2 was moderately differentiated HCC, G3 was poorly differentiated HCC. Through statistical analysis, we found strong CYP17A1 expression in 55% of relatively better differentiated HCC (G1), 27% or 20% in G2 or G3, respectively. This indicates that CYP17A1 expression may be associated with degree of liver cancer malignancy. CYP17A1 expression in different degree of HCC differentiation is shown in FIG. 6.

TABLE 1
Correlation between CYP17A1 expression
and clinical pathological parameters

| | | Number of patients (%) | | |
|---|---|---|---|---|
| | | Weak | Strong | |
| Parameters | Patients | CYP17A1 | CYP17A1 | P value* |
| Tissue | | | | |
| HCC | 87 | 37 (42.5) | 50 (57.5) | <0.001 |
| Adjacent tissue | 87 | 78 (89.7) | 9 (10.3) | |
| Age of patient | | | | |
| <60 | 57 | 42 (73.7) | 15 (26.3) | >0.05 |
| ≧60 | 30 | 19 (63.4) | 11 (36.6) | |
| Gender | | | | |
| Male | 80 | 54 (67.5) | 26 (32.5) | >0.05 |
| Female | 7 | 7 (100) | 0 (0) | |
| Histological grade | | | | |
| G1 | 18 | 8 (44.4) | 10 (55.6) | 0.036 |
| G2 | 40 | 29 (72.5) | 11 (27.5) | |
| G3 | 25 | 20 (80) | 5 (20) | |

*P values were calculated using Chi-square test, P < 0.05 was considered significant.

### Example 10

ELISA Detection of High CYP17A1 Expression in Sera of Liver Cancer Patients 1. ELISA design

Goat anti-human CYP17A1 polyclonal antibody was used as capture antibody, rabbit anti-human CYP17 polyclonal antibody was used as detection antibody. Capture antibody mainly acted on N-terminus of CYP17A1 protein, and detection antibody mainly acted on C-terminus of CYP17A1 protein. Full-length human CYP17A1 recombinant protein was used as standard reference materials to generate standard curve used in ELISA detection. CYP17A1 protein standards were fold-diluted to a gradient with different concentrations (0 pg/ml, 156.25 pg/ml, 312.5 pg/ml, 625 pg/ml, 1250 pg/ml, 2500 pg/ml, 5000 pg/ml, 7500 pg/mi, 10000 pg/ml). OD values were measured to generate standard curve. As shown in standard curve, ELISA system had better sensitivity and accuracy. R², square of correlation coefficient. The closer the value to 1 indicates the higher the accuracy of the curve. (FIGS. 7A and 7B)

2. Analysis and determination of CYP17A1 protein amount in sera of patients with liver cancer, hepatitis, cirrhosis, and lung cancer.

Using the above-mentioned ELISA system, CYP17A1 expression in serum samples of total 212 cases of different people groups were determined, including 115 cases of HCC serum samples, 30 cases of healthy person serum samples, 40 cases of HBV serum samples, 17 cases of cirrhosis serum samples, and 10 cases of lung cancer serum samples.

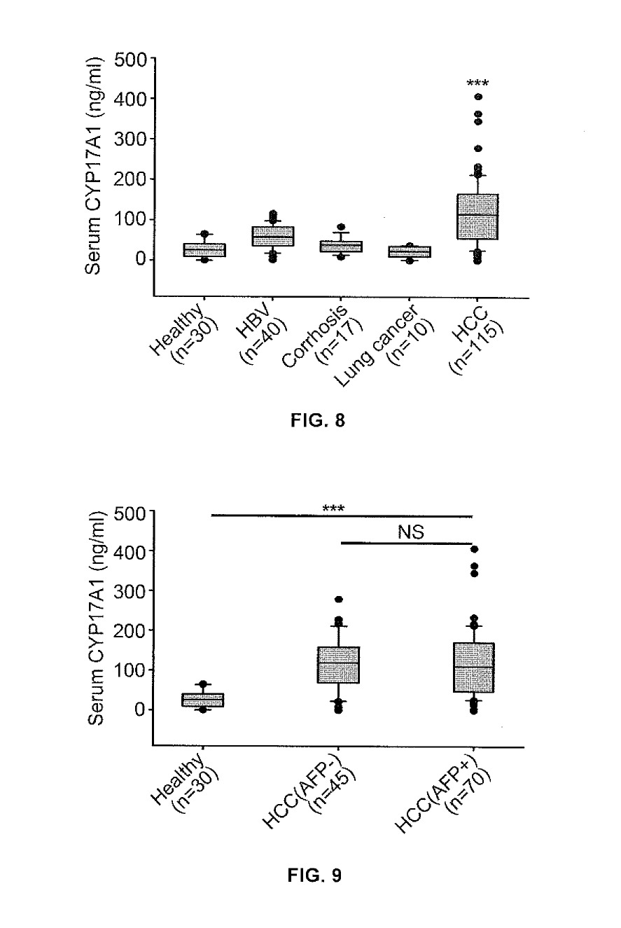

As shown in FIG. 8, CYP17A1 amount in sera of various people groups were all presented as median numbers. The amount in healthy people group was 25.5 ng/ml (variation range 0-65.2 ng/ml), the amount in HBV was 57.7 ng/ml (variation range 1.3-116 ng/ml), the amount in cirrhosis was 39.2 ng/ml (variation range 8.9-83.7 ng/ml), the amount in lung cancer was 22.9 ng/ml (variation range 0.05-37.5 ng/ml). Whereas, the amount in HCC patients was 115.1 ng/ml (variation range 0-407.5 ng/ml). Statistical analysis showed that the amount of CYP17A1 in liver cancer serum samples was significantly higher than in other non-liver cancer serum samples (***, P <0.001).

### Example 11

Analysis and CYP17A1 Protein Amount in Serum Samples of AFP-Negative and AFP-Positive Liver Cancer Patients

Based on 20 ng/ml, which is commonly used in clinic as AFP cut-off point, CYP17A1 amount in serum samples of AFP-negative liver cancer was 119.9 ng/ml (variation range 0-279.3 ng/ml). CYP17A1 amount in serum samples of AFP-positive liver cancer was 111.2 ng/ml (variation range 0-407.5 ng/ml).

Statistical analysis showed that CYP17A1 expression was high in AFP-negative or -positive liver cancer patients and was significantly higher than the amount in healthy persons (25.5 ng/ml, variation range 0-65.2 ng/ml, P <0.001) (FIG. 9). It is worth noting that high CYP17A1 expression was still present in serum samples of AFP-negative liver cancer, indicating that CYP17A1 can supplement detection rate of AFP-negative liver cancer and has special value in clinical diagnosis.

### Example 12

Comparison Between CYP17A1 and AFP As Liver Cancer Diagnostic Reagents

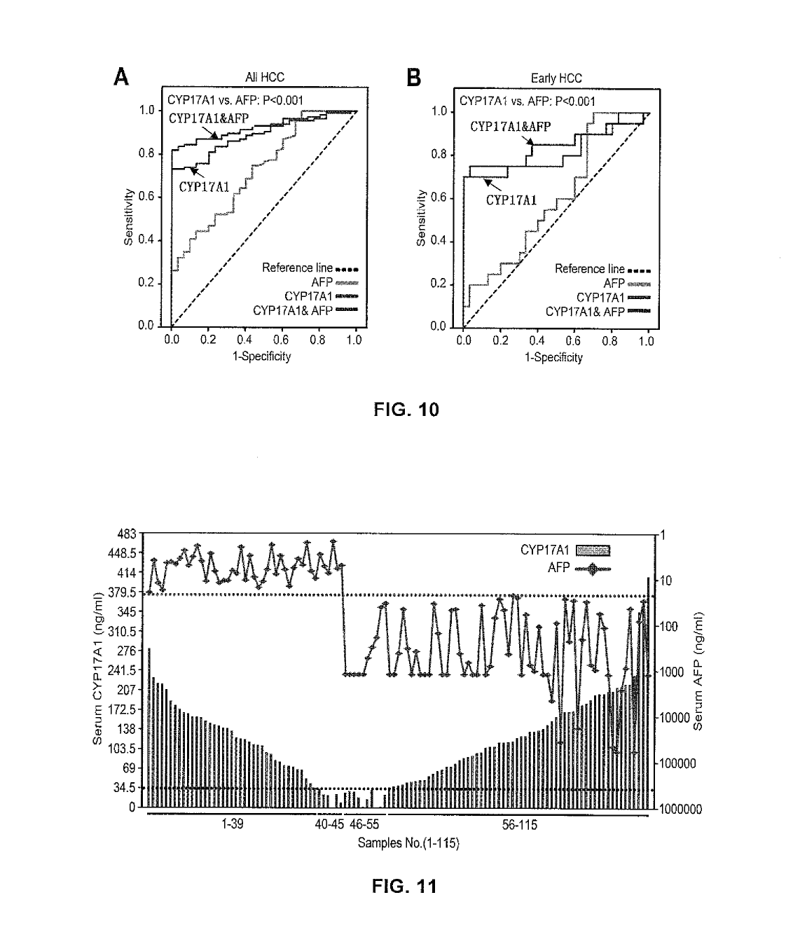

CYP17A1 protein used as indicators of sensitivity and specificity, etc. in liver cancer diagnosis was further analyzed and compared with an existing liver cancer diagnostic marker AFP. Their sensitivity and specificity in distinguishing between liver cancer patients and healthy people group were analyzed through ROC curve. As results shown in FIG. 10A, when distinguishing liver cancer patients from healthy persons, 34.5 ng/ml was used as cut-off point, AUC of CYP17A1 (area under ROC curve, the larger the value, the higher the determination value) was 0.89, corresponding sensitivity and specificity could reach, respectively, 86% and 70%, accuracy rate was 83%. Whereas, in the same samples, AUC of AFP was 0.73, corresponding sensitivity and specificity were, respectively, 61% and 67%, accuracy rate was 62%. CYP17A1 was significantly better than AFP (P <0.001). Combination of the two could further improve sensitivity and specificity to 90% and 70%, accuracy rate was 86%, AUC could be increased to 0.92.

Further, as shown in FIG. 10B, in distinguishing clinical grade I-II early I-ICC from healthy persons, 34.5 ng/ml was used as cut-off point, AUC of CYP17A1 was 0.82, corresponding sensitivity and specificity could reach, respectively, 75% and 67%, accuracy rate was 80%. Whereas, in the same samples, AUC of AFP was 0.60, corresponding sensitivity and specificity were, respectively, 45% and 65%, accuracy rate was 57%. CYP17A1 was significantly better than AFP (P <0.001). Combination of the two could further improve sensitivity and specificity to 75% and 97%, accuracy rate was 88%, AUC could be increased to 0.85.

### Example 13

Analysis of CYP17A1 and AFP Expression in Serum Samples of Liver Cancer

CYP17A1 concentration of 34.5 ng/ml and AFP concentration of 20 ng/ml were used as cut-off points: expression of these two markers in sera of liver cancer is shown in FIG. 11. Sample No. 1-39, CYP17A1 was higher than cut-off point, AFP was within normal range, the ratio was 33.9%. Samples No. 40-45, CYP17A1 and AFP were all within normal range, the ratio was 5.2%. Samples No. 46-55, CYP17A1 was within normal range, AFP was higher than cut-off point, the ratio was 8.7%. Sample No. 56-115, CYP17A1 and AFP were all higher than cut-off points, the ratio was 52.2%. These results show that the ratio of high expression of CYP17A1 in sera of liver cancer patients was greater than AFP. Liver cancer detection rate of CYP17A1 was significantly better than the existing liver cancer diagnostic marker AFP (P <0.001).

All literature mentioned in the present invention are incorporated by reference in the present application, as if each reference were individually incorporated by reference. It should also be understood, after reading the disclosure of the present invention, one skilled in the art could make various changes and modifications to the present invention. These equivalences similarly fall within the scope limited by the appended claims in the present application.

1.-2. (canceled)

3. A diagnostic kit used for the detection of liver cancer, characterized in that, the kit comprises:
(a) an anti-cytochrome p450 family 17 subfamily A polypeptide I (CYP17A1 protein) antibody; and/or
(b) a primer or a pair of primers for specifically amplifying CYP17A1 mRNA or CYP17A1 cDNA.

4. The kit of claim 3, characterized in that, the kit further comprises a label or an instruction, wherein the label or the instruction indicating that the kit is used for the detection or the diagnosis of liver cancer.

5. A method for the detection of liver cancer, wherein the method comprises:
a) preparing a test sample of a subject;
b) detecting an expression level of cytochrome p450 family 17 subfamily A polypeptide 1 gene (CYP17A1) in the test sample, and comparing the detected expression level with a reference value, wherein the expression level of CYP17A1 is higher than the reference value indicating that the subject has liver cancer, or has a higher risk of having liver cancer than a healthy population.

6. The method of claim 5, characterized in that, the test sample is a tissue sample, a blood sample, a serum sample, or a body fluid sample.

7. The method of claim 5, characterized in that, the reference value is an expression level of CYP17A1 in a non-liver cancer sample.

8. The method of claim 5, characterized in that, the detecting step b comprises detecting an amount of CYP17A1 mRNA, or an amount of CYP17A1 cDNA; and/or detecting an amount of CYP17A1 protein.

9. The method of claim 8, characterized in that, the detecting step b comprises using an anti-CYP17A1 protein antibody for the detecting.

10. The method of claim 5, characterized in that, the method further comprises evaluating an expression level of another liver cancer marker in the test sample.

11. The method of claim 6, wherein the test sample is a serum sample.

12. The method of claim 9, characterized in that, the antibody is coupled to or having a detectable marker.

13. The diagnostic kit of claim 3, further comprising a container, wherein the container comprises CYP17A1 protein or the anti-CYP17A1 protein antibody; and a label or an instruction, wherein the label or the instruction indicating that the kit is used for the serum detection or the serum diagnosis of liver cancer.

14. The diagnostic kit of claim 3, further comprises a container, wherein the container comprising the primer or the pair of primers for specifically amplifying CYP17A1 mRNA or CYP17A1 cDNA; and a label or an instruction, wherein the label or the instruction indicating that the kit is used for determining a risk of having liver cancer by quantitative detection of an expression level of CYP17A1.

15. The diagnostic kit of claim 14, wherein the kit is used for the serum detection or the serum diagnosis of liver cancer.

16. A composition for the inhibition of liver cancer cell growth, comprises of an antagonist of cytochrome p450 family 17 subfamily A polypeptide 1 (CYP17A1 protein).

17. The composition of claim 16, wherein the antagonist comprises a CYP17A1-targeting siRNA, an antisense RNA, an antibody, a small molecule compound, or a combination thereof.

18. The diagnostic kit of claim 3, wherein the antibody is coupled to or having a detectable marker.

19. The diagnostic kit of claim 18, wherein the detectable marker is selected from the group: a chromophore, a chemiluminescent group, a fluorophore, an isotope, or an enzyme.

20. The method of claim 12, wherein the detectable marker is selected from the group: a chromophore, a chemiluminescent group, a fluorophore, an isotope, or an enzyme.
